(19) United States
(12) Patent Application Publication
(10) Pub. No.: US 2019/0383234 A1
(43) Pub. Date: Dec. 19, 2019

(54) INTEGRATED ROCKET MOTOR AGING SENSOR

(71) Applicant: GOODRICH CORPORATION, Charlotte, NC (US)

(72) Inventors: Jean C. Rodriguez, Vallejo, CA (US); Joshua David Carter, Fairfield, CA (US)

(73) Assignee: GOODRICH CORPORATION, Charlotte, NC (US)

(21) Appl. No.: 16/012,642

(22) Filed: Jun. 19, 2018

(51) Int. Cl. F02K 9/96; F02K 9/10; F02K 9/34; G01N 33/22

(52) U.S. Cl. CPC F02K 9/96; G01N 33/222; F02K 9/34; F02K 9/10

(57) ABSTRACT

A solid rocket motor propellant grain arrangement may comprise a case, a propellant grain disposed within the case, and an integrated rocket motor aging sensor disposed outward from the propellant grain, wherein the integrated rocket motor aging sensor is configured to measure data corresponding to a plurality of distinct locations of the propellant grain. The integrated rocket motor aging sensor may comprise a resistive screen matrix (RSM).

### FIELD

The present disclosure relates generally to solid rocket motors, and more particularly, to systems and methods for assessing propellant grain lifespan.

### BACKGROUND

Rocket propellant grains rely on a polymer binder for their structural integrity. Structural integrity is described by mechanical properties that determine the propellant grain lifespan. While the chemical composition of a polymer type affects the way it ages, the changes in propellant grain mechanical properties due to polymer aging are a factor in determining propellant grain lifespan. One method of assessing the lifespan of a solid rocket motor is by destructively disassembling the solid rocket motor to measure mechanical properties of the propellant grain.

### SUMMARY

A method for non-destructively determining a health of a solid rocket motor propellant grain is disclosed, comprising receiving, by a controller, a first data corresponding to a plurality of distinct locations of the solid rocket motor propellant grain from an integrated rocket motor aging sensor at a first time, receiving, by the controller, a second data corresponding to the plurality of distinct locations of the solid rocket motor propellant grain from the integrated rocket motor aging sensor at a second time, and comparing, by the controller, the first data with the second data.

In various embodiments, the integrated rocket motor aging sensor comprises a resistive screen matrix (RSM).

In various embodiments, the second data indicates at least one of an expansion or contraction of the solid rocket motor propellant grain.

In various embodiments, the solid rocket motor propellant grain is a solid mass with an exposed inner surface area defining a perforation volume in the interior of the solid rocket motor propellant grain.

In various embodiments, the first data corresponds to a plurality of distinct locations of an outer surface of the solid rocket motor propellant grain.

In various embodiments, receiving the first data comprises receiving a plurality of first datum corresponding to a plurality of nodes of the RSM, wherein each node corresponds to one of the distinct locations.

In various embodiments, receiving the second data comprises receiving a plurality of second datum corresponding to the plurality of nodes of the RSM.

A solid rocket motor propellant grain arrangement is disclosed, comprising a case, a propellant grain disposed within the case, and an integrated rocket motor aging sensor disposed outward from the propellant grain, wherein the integrated rocket motor aging sensor is configured to measure data corresponding to a plurality of distinct locations of the propellant grain.

In various embodiments, the integrated rocket motor aging sensor comprises a resistive screen matrix (RSM).

In various embodiments, the integrated rocket motor aging sensor surrounds an outer surface of the propellant grain.

In various embodiments, the integrated rocket motor aging sensor is wrapped around the propellant grain.

In various embodiments, the solid rocket motor propellant grain arrangement further comprises a liner surrounding the propellant grain.

In various embodiments, the integrated rocket motor aging sensor is disposed between the liner and the case.

In various embodiments, the integrated rocket motor aging sensor is disposed between the liner and the propellant grain.

In various embodiments, the solid rocket motor propellant grain arrangement further comprises a power electronics and control in electronic communication with the integrated rocket motor aging sensor.

In various embodiments, the propellant grain is a solid mass with an exposed inner surface area defining a perforation volume in the interior of the propellant grain.

In various embodiments, the case is manufactured of a metal.

A method for manufacturing a solid rocket motor propellant grain arrangement is disclosed, comprising disposing an integrated rocket motor aging sensor to surround an outer surface of a propellant grain, and disposing a case to surround the integrated rocket motor aging sensor.

In various embodiments, the method further comprises bonding an inner surface of the integrated rocket motor aging sensor to the propellant grain, and bonding an outer surface of the integrated rocket motor aging sensor to the case, wherein the integrated rocket motor aging sensor comprises a resistive screen matrix (RSM).

In various embodiments, the method further comprises disposing a liner to surround the outer surface of the propellant grain, bonding an inner surface of the integrated rocket motor aging sensor to the liner, and bonding an outer surface of the integrated rocket motor aging sensor to the case.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures.

FIG. 1 illustrates a cross section view of a solid rocket motor comprising a propellant grain having a perforation, in accordance with various embodiments;

FIG. 2 schematically illustrates a resistive screen matrix, in accordance with various embodiments;

FIG. 3A and FIG. 3B illustrate a section view of a solid rocket motor propellant grain arrangement, in accordance with various embodiments;

FIG. 4A illustrates an integrated rocket motor aging sensor disposed between a propellant grain and a case, in accordance with various embodiments;

FIG. 4B illustrates an integrated rocket motor aging sensor disposed between a liner for a propellant grain and a case, in accordance with various embodiments;

FIG. 5 illustrates a method for non-destructively determining a health of a solid rocket motor propellant grain, in accordance with various embodiments; and

FIG. 6 illustrates a method for manufacturing a solid rocket motor propellant grain arrangement, in accordance with various embodiments.

### DETAILED DESCRIPTION

The detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that logical, chemical, and mechanical changes may be made without departing from the scope of the disclosure. Thus, the detailed description herein is presented for purposes of illustration only and not of limitation. For example, the steps recited in any of the method or process descriptions may be executed in any order and are not necessarily limited to the order presented.

Furthermore, any reference to singular includes plural embodiments, and any reference to more than one component or step may include a singular embodiment or step. Also, any reference to attached, fixed, connected, or the like may include permanent, removable, temporary, partial, full, and/or any other possible attachment option. Additionally, any reference to without contact (or similar phrases) may also include reduced contact or minimal contact.

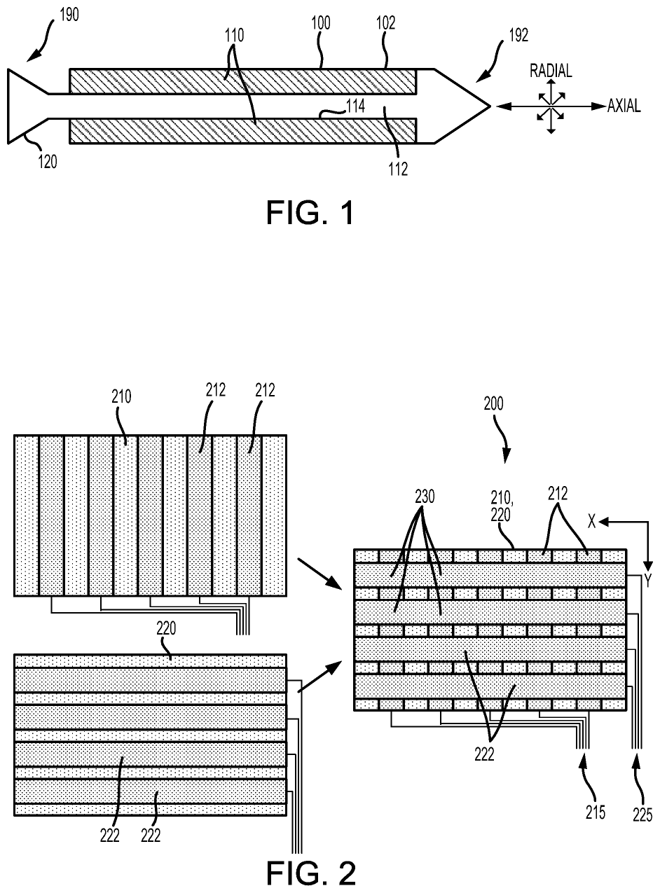

With reference to FIG. 1, a solid rocket motor 100 is illustrated, in accordance with various embodiments. Solid rocket motor 100 may comprise an aft end 190 and a forward end 192. Solid rocket motor 100 may comprise a case 102 extending between aft end 190 and forward end 192. In various embodiments, case 102 may comprise a cylindrical geometry. In various embodiments, case 102 is manufactured of a metal material, such as steel for example. Solid rocket motor 100 may comprise a nozzle 120 disposed at aft end 190. Nozzle 120 may be coupled to case 102. Solid rocket motor 100 may comprise a solid rocket motor propellant grain (propellant grain) 110 disposed within case 102. In various embodiments, propellant grain 110 may be comprised of a composite propellant comprising both a fuel and an oxidizer mixed and immobilized within a cured polymer-based binder. For example, propellant grain 110 may comprise an ammonium nitrate-based composite propellant (ANCP) or ammonium perchlorate-based composite propellant (APCP). Propellant grain 110 may be a solid mass with an exposed inner surface area defining a perforation volume (also referred to herein as a perforation 112) in the interior of the solid rocket motor 100. In this regard, propellant grain 110 may comprise the perforation 112. Perforation 112 may be defined by a bore extending axially through propellant grain 110.

A mechanical property envelope may describe the minimum and maximum performance values for the propellant grain 110 to function as designed. The calculated mechanical property envelope is typically derived from a model produced by the structural analysis of the propellant grain 110 and case 102 geometries. When a propellant grain 110 sample mechanical property falls outside the calculated envelope the propellant grain 110 service life is considered to be at an end.

Typically, in order to determine the health of a plurality of solid rocket motors, a sacrificial solid rocket motor may be disassembled using destructive means to gain access to the propellant of the sacrificial solid rocket motor in order to take proper measurements. The sacrificial solid rocket motor would typically be similar to the plurality of solid rocket motors (e.g., same type, age, storage conditions, etc.). Stated differently, a solid rocket motor may be sacrificed and rendered inoperable in order to estimate the health of a plurality of similarly situated solid rocket motors.

The present disclosure, as described herein, provides systems and methods for non-destructively surveilling solid rocket motor propellant grains for predicting the lifespan and the remaining lifespan of the solid rocket motor.

With reference to FIG. 2, a schematic diagram of an example resistive screen matrix (RSM) 200 is illustrated, in accordance with various embodiments. RSM 200 includes a first substrate 210 and a second substrate 220. The first substrate 210 detects a position of an input point in the X direction, and the second substrate 220 detects the position of the input point in the Y direction. A plurality of first electrodes 212 is formed on the first substrate 210, and a plurality of second electrodes 222 is formed on the second substrate 220. In various embodiments, a spacer layer may be located between the first substrate 210 and the second substrate 220, for separating the plurality of first electrodes 212 and the plurality of second electrodes 222. When the first substrate 210 contacts the second substrate 220, the coordinate values of the input point in the X direction and in the Y direction can be detected according to an electrical resistance of the first electrodes 212 and the second electrodes 222. In this regard, a pressure applied (e.g., by propellant grain 110 with momentary reference to FIG. 1) between the first electrodes 212 and the second electrodes 222 at each node 230 may be proportional to the electrical resistance at each node 230.

In various embodiments, the intersections of first electrodes 212 and second electrodes 222 may define a plurality of nodes 230. In this regard, input corresponding to each node 230 may be measured.

In various embodiments, first electrodes 212 may be formed as strips of electrodes, each strip extending in a first direction (e.g., the Y-direction). In various embodiments, second electrodes 222 may be formed as strips of electrodes, each strip extending in a second direction (e.g., the X-direction). First electrodes 212 may be oriented at a non-zero angle with respect to second electrodes 222. In the illustrated embodiment, first electrodes 212 are oriented at a ninety degree angle with respect to second electrodes 222. However, first electrodes 212 may be oriented at any non-zero degree angle with respect to second electrodes 222. In addition, first electrodes 212 and/or second electrodes 222 may be patterned or non-patterned. Stated differently, first electrodes 212 and/or second electrodes 222 may be uniformly spaced apart from each other or may be randomly placed to form the nodes.

In various embodiments, a first bus 215 may be coupled to first electrodes 212 and a second bus may be coupled to second electrodes 214 whereby the short voltage may be measured.

In various embodiments, first substrate 210 and/or second substrate 220 may be manufactured of an indium tin oxide (ITO) film. In various embodiments, first electrodes 212 and/or second electrodes 222 may be formed by a photo development processes, ITO etching, or etching resist ink. In various embodiments, first electrodes 212 and/or second electrodes 222 can use material such as ITO, indium zinc oxide (IZO), aluminum zinc oxide (AZO), or organic films.

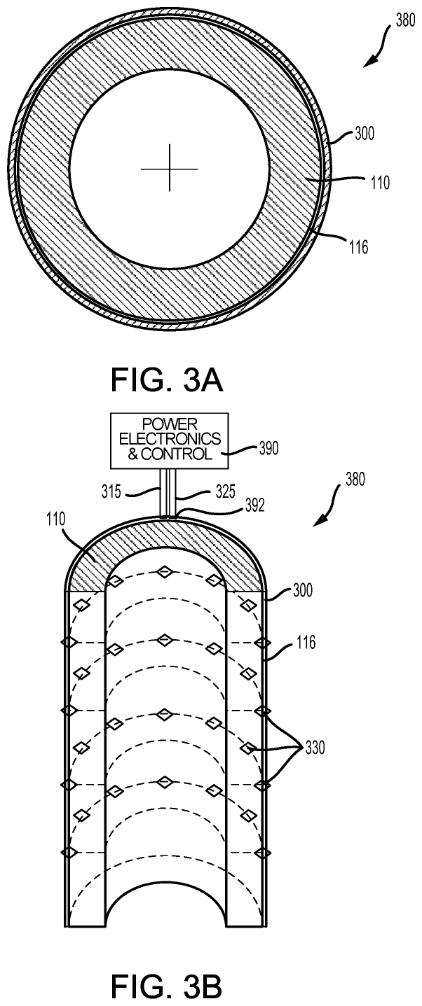

With combined reference to FIG. 3A and FIG. 3B, a solid rocket motor propellant grain arrangement 380 may include an integrated rocket motor aging sensor 300 coupled to an outer surface 116 of propellant grain 110. Integrated rocket motor aging sensor 300 may comprise an RSM. In this regard, integrated rocket motor aging sensor 300 may be similar to RSM 200. In various embodiments, integrated rocket motor aging sensor 300 is wrapped around outer surface 116. Integrated rocket motor aging sensor 300 may define a plurality of nodes (schematically represented by diamonds in FIG. 3B) 330 disposed axially and perimetrically (e.g., circumferentially) along outer surface 116. Integrated rocket motor aging sensor 300 may detect movement (e.g., expansion and/or contraction) of propellant grain 110.

Each node 330 may correspond to a location on outer surface 116 of propellant grain 110. In this regard, integrated rocket motor aging sensor 300 may provide data corresponding to a particular location of propellant grain 110. In particular, integrated rocket motor aging sensor 300 may provide data corresponding to a plurality of pre-determined, distinct locations of propellant grain 110. In this regard, distinct locations of expansion and/or contraction of propellant grain 110 may be measured via integrated rocket motor aging sensor 300 based upon the forces applied by propellant grain 110 to each node of integrated rocket motor aging sensor 300. In this regard, integrated rocket motor aging sensor 300 may provide a “map” of expansion and/or contraction of propellant grain 110, for example, in the form of a data output file, such as, for example, a database table, a delimited format such as a comma-separated values (CSV) file, or any other suitable data structure.

A power electronics and control 390 may be in electronic communication with integrated rocket motor aging sensor 300. Power electronics and control 390 may comprise a processor. Power electronics and control 390 may comprise a tangible, non-transitory memory for receiving data from integrated rocket motor aging sensor 300 via a first bus 315 and a second bus 325. In various embodiments, first bus 315 and second bus 325 may be similar to first bus 215 and second bus 225, respectively, with momentary reference to FIG. 2.

Power electronics and control 390 may provide electric power to integrated rocket motor aging sensor 300. In various embodiments, power electronics and control 390 may be in continuous electronic communication with integrated rocket motor aging sensor 300 for active health monitoring. In various embodiments, power electronics and control 390 may be removably coupled to integrated rocket motor aging sensor 300, for example via connectors 392, for passive health monitoring, e.g., snapshots in time.

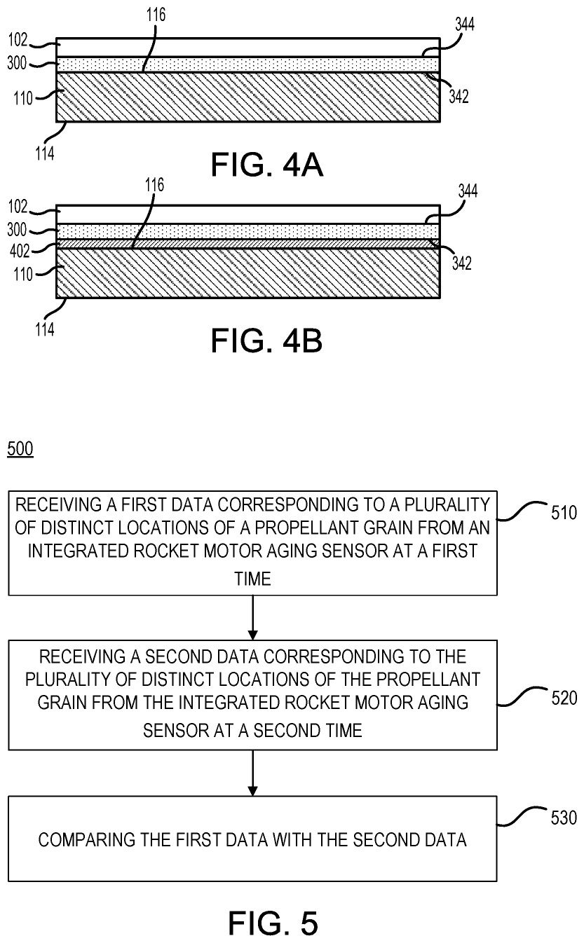

With reference to FIG. 4A, integrated rocket motor aging sensor 300 may be disposed between propellant grain 110 and case 102. Integrated rocket motor aging sensor 300 may be bonded to propellant grain 110. Integrated rocket motor aging sensor 300 may be bonded to case 102. In this regard, an inner surface (e.g., an inner diameter (ID) surface) 342 of integrated rocket motor aging sensor 300 may be bonded to propellant grain 110, and an outer surface (e.g., an outer diameter (OD) surface) 344 of integrated rocket motor aging sensor 300 may be bonded to case 102. Integrated rocket motor aging sensor 300 may be sensitive to movement of propellant grain 110 relative to case 102 in response to both inner surface 342 being bond to propellant grain 110 and outer surface 344 being bonded to case 102. For example, a first substrate (e.g., first substrate 210 of FIG. 2) of integrated rocket motor aging sensor 300 may be bonded to propellant grain 110, and a second substrate (e.g., second substrate 220 of FIG. 2) of integrated rocket motor aging sensor 300 may be bonded to case 102.

With reference to FIG. 4B, a liner 402 may surround propellant grain 110. Liner 402 may protect propellant grain 110, for preventing undesirable combustion of propellant grain 110. In various embodiments, liner 402 may be manufactured of the same material as propellant grain 110, except that oxidizers are omitted from liner 402 to minimize the combustibility of liner 402. Integrated rocket motor aging sensor 300 may be disposed between liner 402 and case 102. Integrated rocket motor aging sensor 300 may be bonded to liner 402. In various embodiments, integrated rocket motor aging sensor 300 may be bonded to liner 402 during a curing process of liner 402. Integrated rocket motor aging sensor 300 may be bonded to case 102. In this regard, inner surface 342 of integrated rocket motor aging sensor 300 may be bonded to liner 402, and outer surface 344 of integrated rocket motor aging sensor 300 may be bonded to case 102. Integrated rocket motor aging sensor 300 may be sensitive to movement of propellant grain 110 relative to case 102 in response to both inner surface 342 being bond to liner 402 and outer surface 344 being bonded to case 102. For example, a first substrate (e.g., first substrate 210 of FIG. 2) of integrated rocket motor aging sensor 300 may be bonded to liner 402, and a second substrate (e.g., second substrate 220 of FIG. 2) of integrated rocket motor aging sensor 300 may be bonded to case 102.

In various embodiments, integrated rocket motor aging sensor 300 may be manufactured of material that is sufficiently flexible to handle expansion and/or contraction of propellant grain 110 without breaking. For example, cracking or fractures in integrated rocket motor aging sensor 300 could cause an open circuit condition causing integrated rocket motor aging sensor 300 to fail.

With reference to FIG. 5, a method 500 for determining a health of a solid rocket motor propellant grain is illustrated, in accordance with various embodiments. Method 500 includes receiving a first data corresponding to a plurality of distinct locations of a propellant grain from an integrated rocket motor aging sensor at a first time (step 510). Method 500 includes receiving a second data corresponding to the plurality of distinct locations of the propellant grain from the integrated rocket motor aging sensor at a second time (step 520). Method 500 includes comparing the first data with the second data (step 530).

With combined reference to FIG. 3B, and FIG. 5, step 510 may include receiving, by power electronics and control 390, data from integrated rocket motor aging sensor 300 corresponding to each node 330. The data may be in the form of a resistance value for each node 330. The data may be in the form of a voltage value for each node 330. In this regard, one datum for each node 330 may be received. In this regard, the data may be stored in a vector or array of vectors. In this regard, each datum corresponds to the location of propellant grain 110 of each node. Step 520 may be similar to step 510, except that step 520 is performed at a later time (i.e., a second time) from step 510 to obtain a second data from integrated rocket motor aging sensor 300 corresponding to each node 330. For example, step 520 may be performed days, weeks, months, or years later from step 510. Step 530 may include comparing the second data with the first data. Step 530 may include determining a difference between the first data and the second data. For example, a plurality of differences between the first data and the second data may be calculated for each node 330. In this regard, expansion and/or contraction of propellant grain 110 may be detected at a plurality of distinct locations along the outer surface 116 of propellant grain 110. The data may be compared with models, thresholds values, or the like for determining a health or lifespan of the propellant grain 110.

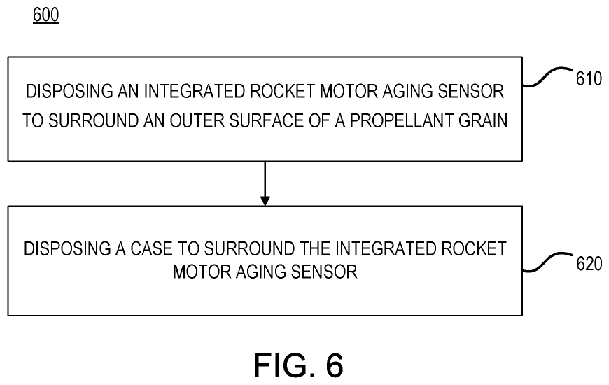

With reference to FIG. 6, a method 600 for manufacturing a solid rocket motor propellant grain arrangement is illustrated, in accordance with various embodiments. Method 600 includes disposing an integrated rocket motor aging sensor to surround an outer surface of a propellant grain (step 610). Method 600 includes disposing a case to surround the integrated rocket motor aging sensor (step 620).

With combined reference to FIG. 4A and FIG. 6, step 610 may include disposing integrated rocket motor aging sensor 300 to surround outer surface 116 of a propellant grain 110. Step 610 may include bonding inner surface 342 of integrated rocket motor aging sensor 300 to propellant grain 110. Step 620 may include disposing case 102 to surround integrated rocket motor aging sensor 300. Step 620 may include bonding outer surface 344 of integrated rocket motor aging sensor 300 to case 102.

With combined reference to FIG. 4B and FIG. 6, step 610 may include disposing liner 402 to surround outer surface 116 of a propellant grain 110. Step 610 may include disposing integrated rocket motor aging sensor 300 to surround liner 402. Step 610 may include bonding inner surface 342 of integrated rocket motor aging sensor 300 to liner 402.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods and apparatus are provided herein. In the detailed description herein, references to “one embodiment”, “an embodiment”, “various embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to invoke 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method for non-destructively determining a health of a solid rocket motor propellant grain, comprising:
receiving, by a controller, a first data corresponding to a plurality of distinct locations of the solid rocket motor propellant grain from an integrated rocket motor aging sensor at a first time;
receiving, by the controller, a second data corresponding to the plurality of distinct locations of the solid rocket motor propellant grain from the integrated rocket motor aging sensor at a second time; and
comparing, by the controller, the first data with the second data.

2. The method of claim 1, wherein the integrated rocket motor aging sensor comprises a resistive screen matrix (RSM).

3. The method of claim 2, wherein the second data indicates at least one of an expansion or contraction of the solid rocket motor propellant grain.

4. The method of claim 2, wherein the solid rocket motor propellant grain is a solid mass with an exposed inner surface area defining a perforation volume in the interior of the solid rocket motor propellant grain.

5. The method of claim 2, wherein the first data corresponds to the plurality of distinct locations of an outer surface of the solid rocket motor propellant grain.

6. The method of claim 2, wherein receiving the first data comprises receiving a plurality of first datum corresponding to a plurality of nodes of the RSM, wherein each node corresponds to at least one of the plurality of distinct locations.

7. The method of claim 6, wherein receiving the second data comprises receiving a plurality of second datum corresponding to the plurality of nodes of the RSM.

8. A solid rocket motor propellant grain arrangement, comprising:
a case;
a propellant grain disposed within the case; and
an integrated rocket motor aging sensor disposed outward from the propellant grain, wherein the integrated rocket motor aging sensor is configured to measure data corresponding to a plurality of distinct locations of the propellant grain.

9. The solid rocket motor propellant grain arrangement of claim 8, wherein the integrated rocket motor aging sensor comprises a resistive screen matrix (RSM).

10. The solid rocket motor propellant grain arrangement of claim 9, wherein the integrated rocket motor aging sensor surrounds an outer surface of the propellant grain.

11. The solid rocket motor propellant grain arrangement of claim 10, wherein the integrated rocket motor aging sensor is wrapped around the propellant grain.

12. The solid rocket motor propellant grain arrangement of claim 9, further comprising a liner surrounding the propellant grain.

13. The solid rocket motor propellant grain arrangement of claim 12, wherein the integrated rocket motor aging sensor is disposed between the liner and the case.

14. The solid rocket motor propellant grain arrangement of claim 12, wherein the integrated rocket motor aging sensor is disposed between the liner and the propellant grain.

15. The solid rocket motor propellant grain arrangement of claim 9, further comprising a power electronics and control in electronic communication with the integrated rocket motor aging sensor.

16. The solid rocket motor propellant grain arrangement of claim 9, wherein the propellant grain is a solid mass with an exposed inner surface area defining a perforation volume in the interior of the propellant grain.

17. The solid rocket motor propellant grain arrangement of claim 9, wherein the case is manufactured of a metal.

18. A method for manufacturing a solid rocket motor propellant grain arrangement, comprising:
disposing an integrated rocket motor aging sensor to surround an outer surface of a propellant grain; and
disposing a case to surround the integrated rocket motor aging sensor.

19. The method of claim 18, further comprising:
bonding an inner surface of the integrated rocket motor aging sensor to the propellant grain; and
bonding an outer surface of the integrated rocket motor aging sensor to the case, wherein the integrated rocket motor aging sensor comprises a resistive screen matrix (RSM).

20. The method of claim 18, further comprising:
disposing a liner to surround the outer surface of the propellant grain;
bonding an inner surface of the integrated rocket motor aging sensor to the liner; and
bonding an outer surface of the integrated rocket motor aging sensor to the case.
